(19) United States
(12) Patent Application Publication
(10) Pub. No.: US 2007/0149428 A1
(43) Pub. Date: Jun. 28, 2007

(54) Method of Packaging a Lens

(75) Inventors: Daniel M. Ammon JR., Penfield, NY (US); Jay F. Kunzler, Canandaigua, NY (US); Joseph C. Salamone, Boca Raton, FL (US); Susan E. Burke, Batavia, NY (US); Ronald J. Koch, Webster, NY (US); Jeffrey G. Linhardt, Fairport, NY (US)

Correspondence Address: Bausch & Lomb Incorporated, One Bausch & Lomb Place, Rochester, NY, 14604-2701, US

(73) Assignee: BAUSCH & LOMB INCORPORATED, Rochester, NY (US)

(21) Appl. No.: 11/610,372

(22) Filed: Dec. 13, 2006

Related U.S. Application Data
(60) Provisional application No. 60/750,238, filed on Dec. 14, 2005.

(51) Int. Cl. A61L 2/00; C08K 5/00

(52) U.S. Cl. 510/112; 206/5.1

(57) ABSTRACT

A contact lens package includes a sealed receptacle containing a contact lens immersed in a sterile solution. The contact lens is made of a silicone hydrogel copolymer, and the solution includes a stabilizing agent in an amount effective to inhibit changes in physical properties of the silicone hydrogel copolymer.

This application claims the benefit under 35 USC 119(e) of provisional patent application Ser. No. 60/750,238, filed Dec. 14, 2005.

### FIELD OF THE INVENTION

This invention relates to contact lens packages containing a silicone hydrogel contact lens having improved stability and shelf life.

### BACKGROUND OF THE INVENTION

Silicone hydrogels represent one class of materials used for contact lens applications. Hydrogels comprise a hydrated, crosslinked polymeric system containing water in an equilibrium state. In the case of silicone hydrogel contact lenses, the hydrogel copolymers are generally prepared by polymerizing a monomeric mixture containing at least one lens-forming silicone-containing monomer and at least one lens-forming hydrophilic monomer.

Hydrogel contact lenses are typically packaged in a glass vial or plastic blister package that includes a receptacle portion to hold the contact lens and a sterile packaging solution. This vial or receptacle, containing the contact lens immersed in the solution, is hermetically sealed, for example, by sealing lidstock on the package over the receptacle. The package serves as a means to safely ship and store the lens. A contact lens may not be removed from its package for use for quite some time. For example, lenses in their package may be held in inventory by a manufacturer or a distributor; also, a contact lens wearer may purchase and receive a long-term supply of lenses. Accordingly, it is important that the packaged lenses have sufficient shelf life. In fact, contact lens packages will indicate an expiration date indicating the end of the shelf life of the lens.

Silicone hydrogel copolymers have a greater tendency than conventional, non-silicone hydrogels lenses to be hydrolytically unstable. Stated differently, silicone hydrogel contact lenses have a greater tendency to undergo a change in mechanical properties, such as modulus, over time, due to changes in crosslinking density of the hydrogel copolymer while the lens is packaged and stored. Such changes in mechanical properties may translate to a shorter shelf life than desired. Thus, it is not uncommon for silicone hydrogel contact lenses to have a shorter shelf life than conventional, non-silicone hydrogel lenses.

### SUMMARY OF THE INVENTION

This invention recognized the aforementioned problems and solves the various problems associated with packaging and storing silicone hydrogel contact lenses.

According to various aspects, this invention provides a contact lens package including a sealed receptacle containing a contact lens immersed in a sterile solution, wherein the contact lens is made of a silicone hydrogel copolymer, and the solution comprises a stabilizing agent in an amount effective to inhibit changes in physical properties of the silicone hydrogel copolymer.

According to various preferred embodiments, the stabilizing agent inhibits changes in mechanical properties, such as changes in modulus.

The stabilizing agent may form ionic complexes or hydrogen bonding complexes with the silicone hydrogel copolymer.

As an example, the silicone hydrogel copolymer is anionic and the stabilizing agent contains a cationic charge, including a cationic agent and a zwitterionic agent.

As an example, the silicone hydrogel copolymer is cationic and the stabilizing agent contains an anionic charge, including an anionic agent and a zwitterionic agent.

As an example, the stabilizing agent is an amine that complexes with anionic groups of the copolymer. The amine moiety of the stabilizing agent and/or copolymer may be quaternized ammonium.

As an example, the stabilizing agent contains groups that hydrogen bond with hydrogen bond accepting groups of the copolymer.

According to preferred embodiments, lidstock is sealed over the receptacle containing the solution and the contact lens, and the contact lens and solution are sterilized in the sealed receptacle, such as by autoclaving.

This invention also provides a method comprising: sealing a receptacle of a contact lens package that contains a solution and a contact lens, wherein the contact lens is made of a silicone hydrogel copolymer; and storing the contact lens in the package for an extended period of time, wherein the stabilizing agent inhibits changes in physical properties of the silicone hydrogel copolymer during storage.

This invention provides a method of improving the hydrolytic stability of a contact lens made of a silicone hydrogel copolymer, comprising storing the contact lens in a sealed package and immersed in a sterile solution comprising a stabilizing agent, wherein the stabilizing agent inhibits changes in physical properties of the silicone hydrogel copolymer during storage.

This invention provides a method of increasing the shelf life of a contact lens made of a silicone hydrogel copolymer and contained in a sealed package, comprising storing the contact lens in the sealed package and immersed in a solution comprising a stabilizing agent that inhibits changes in physical properties of the silicone hydrogel copolymer during storage.

This invention includes a method of providing a silicone hydrogel contact lens with a shelf life of at least 2 years, more preferably at least 3 years, comprising storing the contact lens in the sealed package and immersed in a solution comprising a stabilizing agent that inhibits changes in physical properties of the silicone hydrogel copolymer during storage.

### DETAILED DESCRIPTION OF VARIOUS PREFERRED EMBODIMENTS

This invention is useful for packaging silicone hydrogel contact lenses. Hydrogels comprise a hydrated, crosslinked polymeric system containing water in an equilibrium state. Accordingly, hydrogels are copolymers prepared from hydrophilic monomers. In the case of silicone hydrogels, the hydrogel copolymers are generally prepared by polymerizing a mixture containing at least one lens-forming silicone-containing monomer and at least one lens-forming hydrophilic monomer. Either the silicone-containing monomer or the hydrophilic monomer may function as a crosslinking agent (a crosslinking agent being defined as a monomer having multiple polymerizable functionalities), or alternately, a separate crosslinking agent may be employed in the initial monomer mixture from which the hydrogel copolymer is formed. (As used herein, the term “monomer” or “monomeric” and like terms denote relatively low molecular weight compounds that are polymerizable by free radical polymerization, as well as higher molecular weight compounds also referred to as “prepolymers”, “macromonomers”, and related terms.) Silicone hydrogels typically have a water content between about 10 to about 80 weight percent. In the case where the silicone hydrogel copolymer is formed from a silicone-containing crosslinking agent, the initial monomeric mixture may further comprise a monofunctional silicone-containing monomer.

Examples of useful lens-forming hydrophilic monomers include: amides such as N,N-dimethylacrylamide and N,N-dimethylmethacrylamide; cyclic lactams such as N-vinyl-2-pyrrolidone; (meth)acrylated alcohols, such as 2-hydroxyethyl methacrylate, 2-hydroxyethyl acrylate and glyceryl methacrylate; (meth)acrylated poly(ethylene glycol)s; (meth)acrylic acids such as methacrylic acid and acrylic acid; and azlactone-containing monomers, such as 2-isopropenyl-4,4-dimethyl-2-oxazolin-5-one and 2-vinyl-4,4-dimethyl-2-oxazolin-5-one. (As used herein, the term “(meth)” denotes an optional methyl substituent. Thus, terms such as “(meth)acrylate” denotes either methacrylate or acrylate, and “(meth)acrylic acid” denotes either methacrylic acid or acrylic acid.) Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277, the disclosures of which are incorporated herein by reference. Other suitable hydrophilic monomers will be apparent to one skilled in the art.

Applicable silicone-containing monomeric materials for use in the formation of silicone hydrogels are well known in the art and numerous examples are provided in U.S. Pat. Nos. 4,136,250; 4,153,641; 4,740,533; 5,034,461; 5,070,215; 5,260,000; 5,310,779; and 5,358,995.

Examples of applicable silicone-containing monomers include bulky polysiloxanylalkyl (meth)acrylic monomers. An example of such monofunctional, bulky polysiloxanylalkyl (meth)acrylic monomers are represented by the following Formula I:

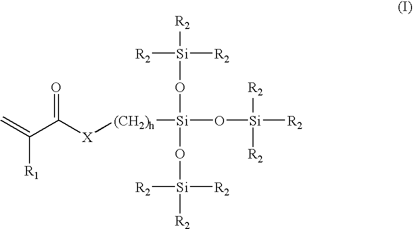

wherein:

X denotes —O— or —NR—;

each R₁ independently denotes hydrogen or methyl;
each R₂ independently denotes a lower alkyl radical, phenyl radical or a group represented by

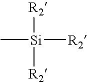

wherein each R₂′ independently denotes a lower alkyl or phenyl radical; and h is 1 to 10. One preferred bulky monomer is 3-methacryloxypropyltris(trimethyl-siloxy)silane or tris(trimethylsiloxy)silylpropyl methacrylate.

Another class of representative silicone-containing monomers includes silicone-containing vinyl carbonate or vinyl carbamate monomers such as: 1,3-bis[4-vinyloxycarbonyloxy)but-1-yl]tetramethyldisiloxane; 1,3-bis[4-vinyloxycarbonyloxy)but-1-yl]polydimethylsiloxane; 3-(trimethylsilyl)propyl vinyl carbonate; 3-(vinyloxycarbonylthio)propyl[tris(trimethylsiloxy)silane]; 3-[tris(trimethylsiloxy)silyl]propyl vinyl carbamate; 3-[tris(trimethylsiloxy)silyl]propyl allyl carbamate; 3-[tris(trimethylsiloxy)silyl]propyl vinyl carbonate; t-butyldimethylsiloxyethyl vinyl carbonate; trimethylsilylethyl vinyl carbonate; and trimethylsilylmethyl vinyl carbonate.

An example of silicone-containing vinyl carbonate or vinyl carbamate monomers are represented by Formula II: 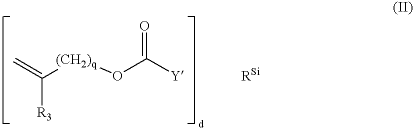
wherein:

Y′ denotes —O—, —S— or —NH—;

R^{Si} denotes a silicone-containing organic radical;

R₃ denotes hydrogen or methyl;

d is 1, 2, 3 or 4; and q is 0 or 1.

Suitable silicone-containing organic radicals R^{Si} include the following: 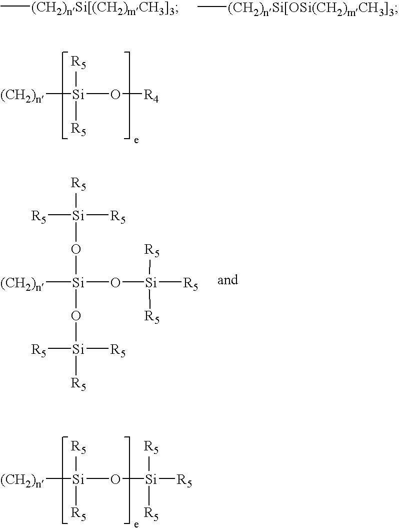
wherein:

R₄ denotes 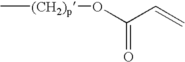
wherein p′ is 1 to 6;

R₅ denotes an alkyl radical or a fluoroalkyl radical having 1 to 6 carbon atoms;

e is 1 to 200; n′ is 1, 2, 3 or 4; and m′ is 0, 1, 2, 3, 4 or 5.

An example of a particular species within Formula II is represented by Formula III:

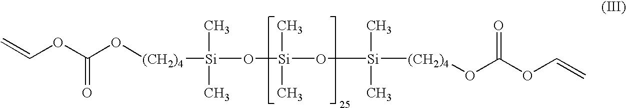

Another class of silicone-containing monomers includes polyurethane-polysiloxane macromonomers (also sometimes referred to as prepolymers), which may have hard-soft-hard blocks like traditional urethane elastomers. Examples of silicone urethane monomers are represented by Formulae IV and V:
E(*D*A*D*G)ₐ*D*A*D*E′; or (IV)
E(*D*G*D*A)ₐ*D*G*D*E′; (V)
wherein:

D denotes an alkyl diradical, an alkyl cycloalkyl diradical, a cycloalkyl diradical, an aryl diradical or an alkylaryl diradical having 6 to 30 carbon atoms;

G denotes an alkyl diradical, a cycloalkyl diradical, an alkyl cycloalkyl diradical, an aryl diradical or an alkylaryl diradical having 1 to 40 carbon atoms and which may contain ether, thio or amine linkages in the main chain;

* denotes a urethane or ureido linkage;

a is at least 1;

A denotes a divalent polymeric radical of Formula VI: 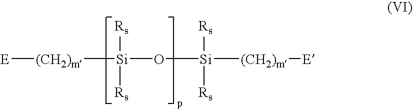
wherein:

each R_{S} independently denotes an alkyl or fluoro-substituted alkyl group having 1 to 10 carbon atoms which may contain ether linkages between carbon atoms;

m′ is at least 1; and

p is a number which provides a moiety weight of 400 to 10,000;

each of E and E′ independently denotes a polymerizable unsaturated organic radical represented by Formula VII: 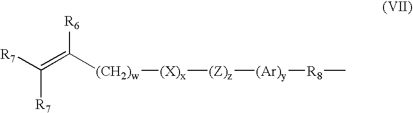
wherein:

R₆ is hydrogen or methyl;

R₇ is hydrogen, an alkyl radical having 1 to 6 carbon atoms, or a —CO—Y—R₉ radical wherein Y is —O—, —S— or —NH—;

R₈ is a divalent alkylene radical having 1 to 10 carbon atoms;

R₉ is a alkyl radical having 1 to 12 carbon atoms;

X denotes —CO— or —OCO—;

Z denotes —O— or —NH—;

Ar denotes an aromatic radical having 6 to 30 carbon atoms;

w is 0 to 6; x is 0 or 1; y is 0 or 1; and z is 0 or 1.

A more specific example of a silicone-containing urethane monomer is represented by Formula (VIII):

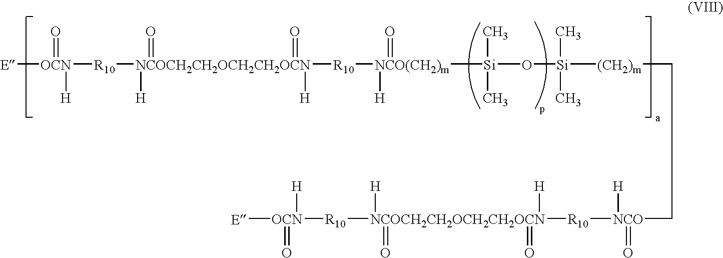

wherein m is at least 1 and is preferably 3 or 4, a is at least 1 and preferably is 1, p is a number which provides a moiety weight of 400 to 10,000 and is preferably at least 30, R₁₀ is a diradical of a diisocyanate after removal of the isocyanate group, such as the diradical of isophorone diisocyanate, and each E″ is a group represented by:

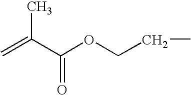

A representative silicone hydrogel material comprises (based on the initial monomer mixture that is copolymerized to form the hydrogel copolymeric material) 5 to 50 percent, preferably 10 to 25, by weight of one or more silicone macromonomers, 5 to 75 percent, preferably 30 to 60 percent, by weight of one or more polysiloxanylalkyl (meth)acrylic monomers, and 10 to 50 percent, preferably 20 to 40 percent, by weight of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 to Deichert et al. discloses additional unsaturated groups, including acryloxy or methacryloxy. Fumarate-containing materials such as those taught in U.S. Pat. Nos. 5,512,205; 5,449,729; and 5,310,779 to Lai are also useful substrates in accordance with the invention. Preferably, the silane macromonomer is a silicone-containing vinyl carbonate or vinyl carbamate or a polyurethane-polysiloxane having one or more hard-soft-hard blocks and end-capped with a hydrophilic monomer.

Specific examples of contact lens materials for which the present invention is useful are taught in U.S. Pat. Nos.: 6,891,010 (Kunzler et al.); 5,908,906 (Kunzler et al.); 5,714,557 (Kunzler et al.); 5,710,302 (Kunzler et al.); 5,708,094 (Lai et al.); 5,616,757 (Bambury et al.); 5,610,252 (Bambury et al.); 5,512,205 (Lai); 5,449,729 (Lai); 5,387,662 (Kunzler et al.); 5,310,779 (Lai); and 5,260,000 (Nandu et al.), the disclosures of which are incorporated herein by reference.

Representative examples of applicable cationic silicon-containing monomeric units include cationic monomers of formula IX: 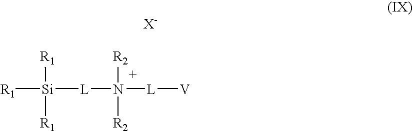
wherein each L independently can be an urethane, carbonate, carbamate, carboxyl ureido, sulfonyl, straight or branched C₁-C₃₀ alkyl group, straight or branched C₁-C₃₀ fluoroalkyl group, ester-containing group, ether-containing group, polyether-containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, substituted or unsubstituted C₆-C₃₀ heteroarylalkyl group, C₅-C₃₀ fluoroaryl group, or hydroxyl substituted alkyl ether and combinations thereof.

X⁻ is at least a single charged counter ion. Examples of single charge counter ions include the group consisting of Cl⁻, Br⁻, I⁻, CF₃CO₂⁻, CH₃CO₂⁻, HCO₃⁻, CH₃SO₄⁻, p-toluenesulfonate, HSO₄⁻, H₂PO₄⁻, NO₃⁻, and CH₃CH(OH)CO₂⁻. Examples of dual charged counter ions would include SO₄²⁻, CO₃²⁻ and HPO₄²⁻. Other charged counter ions would be obvious to one of ordinary skill in the art. It should be understood that a residual amount of counterion may be present in the hydrated product. Therefore, the use of toxic counterions is to be discouraged. Likewise, it should be understood that, for a singularly charged counterion, the ratio of counterion and quaternary siloxanyl will be 1:1. Counterions of greater negative charge will result in differing ratios based upon the total charge of the counterion.

R₁ and R₂ are each independently hydrogen, a straight or branched C₁-C₃₀ alkyl group, straight or branched C₁-C₃₀ fluoroalkyl group, C₁-C₂₀ ester group, ether containing group, polyether containing group, ureido group, amide group, amine group, substituted or unsubstituted C₁-C₃₀ alkoxy group, substituted or unsubstituted C₃-C₃₀ cycloalkyl group, substituted or unsubstituted C₃-C₃₀ cycloalkylalkyl group, substituted or unsubstituted C₃-C₃₀ cycloalkenyl group, substituted or unsubstituted C₅-C₃₀ aryl group, substituted or unsubstituted C₅-C₃₀ arylalkyl group, substituted or unsubstituted C₅-C₃₀ heteroaryl group, substituted or unsubstituted C₃-C₃₀ heterocyclic ring, substituted or unsubstituted C₄-C₃₀ heterocyclolalkyl group, a substituted or unsubstituted C₆-C₃₀ heteroarylalkyl group, fluorine group, a C₅-C₃₀ fluoroaryl group, or a hydroxyl group and V is independently a polymerizable ethylenically unsaturated organic radical.

Monomers of formula IX include those represented by formula X below: 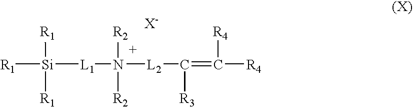
wherein each R₁ is the same and is —OSi(CH₃)₃, R₂ is methyl, L₁ is an alkyl amide, L₂ is a alkyl amide or ester having 2 or 3 carbon atoms that is joined to a polymerizable vinyl group, R₃ is methyl, R₄ is H and X⁻ is Br⁻ or Cl⁻.

Further structures have the following structural formulae XI-XV:

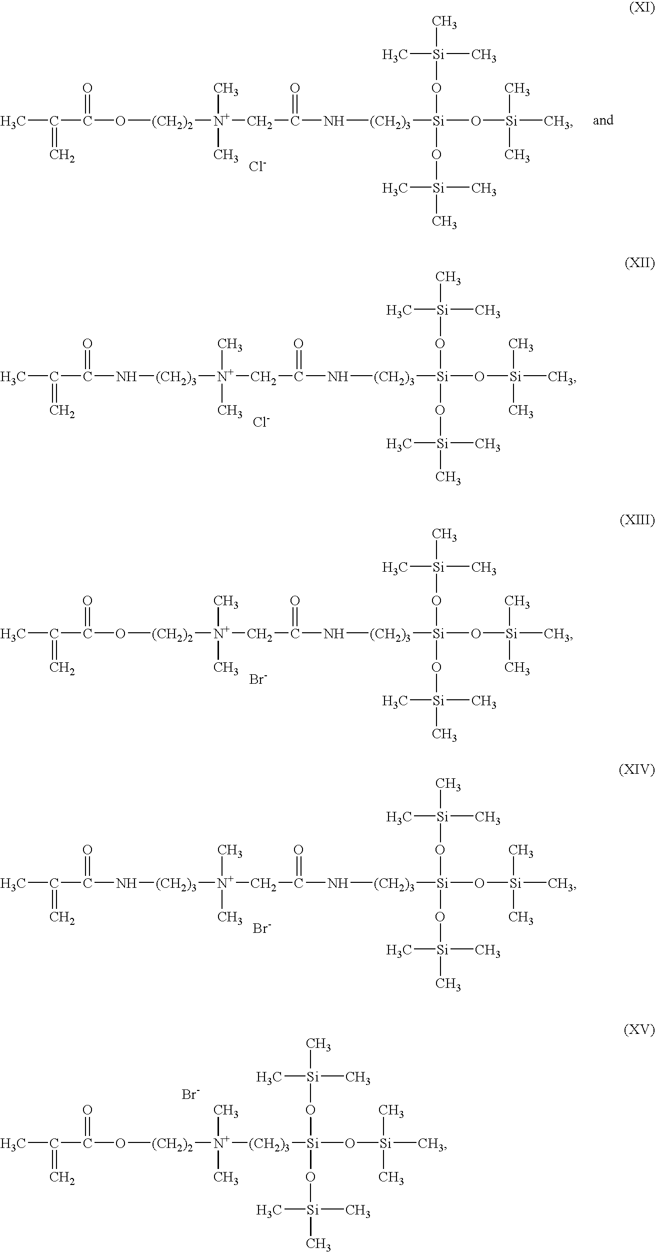

A schematic representation of synthetic methods for making a cationic silicon-containing monomer as disclosed hereinabove is provided below:

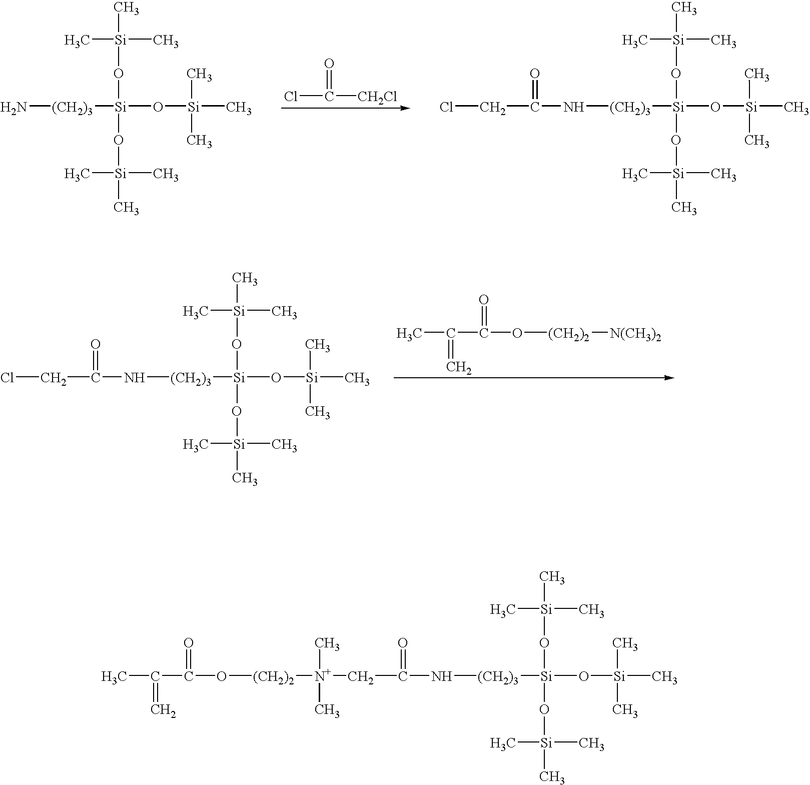

Another class of examples of applicable cationic silicon-containing monomenic units for use herein include cationic monomers of formula XVI: 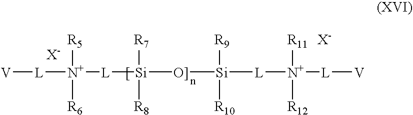
wherein each L can be the same or different and is as defined above for L in formula IX; X⁻ is at least a single charged counter ion as defined above for X⁻ in formula I; R₅, R₆, R₇, R₈, R₉, R₁₀, R₁₁ and R₁₂ are each independently as defined above for R₁ in formula I; V is independently a polymerizable ethylenically unsaturated organic radical and n is an integer of 1 to about 300.

Monomers of formula XVI include those represented by formulae IX-XXI below:

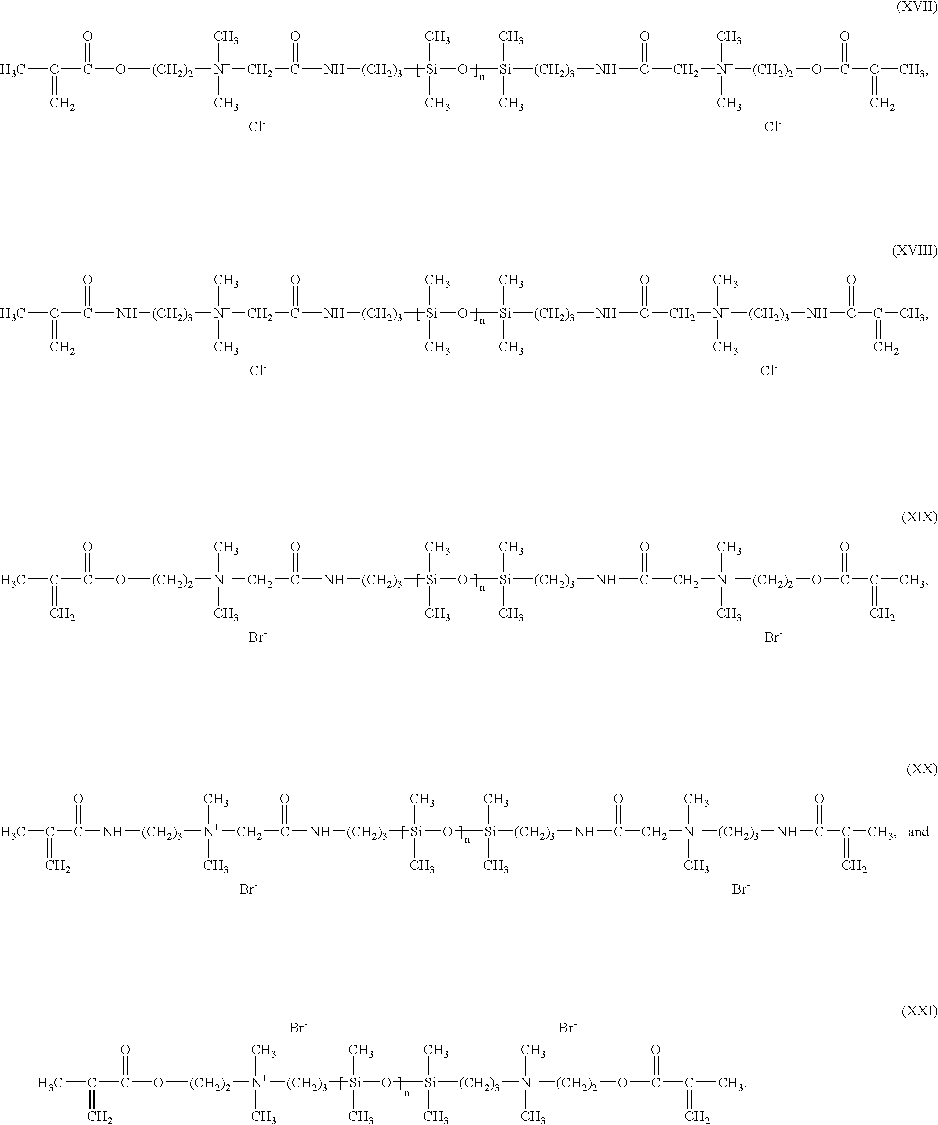

A schematic representation of a synthetic method for making the cationic silicon-containing monomers of formula XVI is provided below:

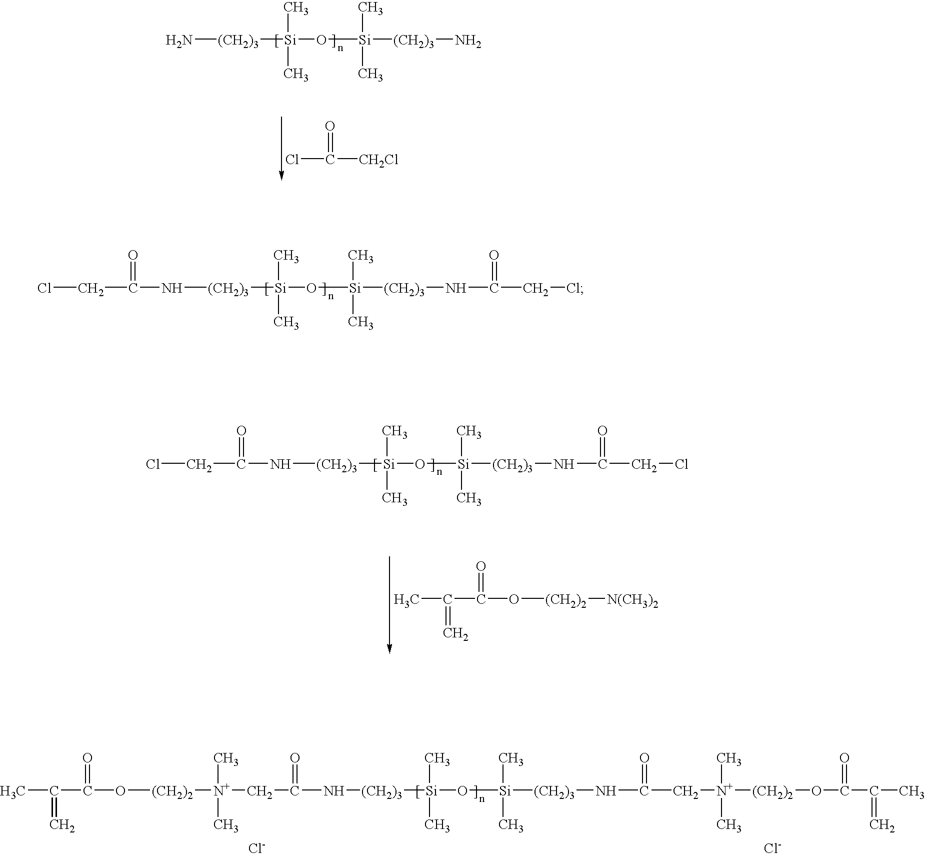

A schematic representation of a synthetic method for making the cationic silicon-containing monomers of formula XIX is provided below:

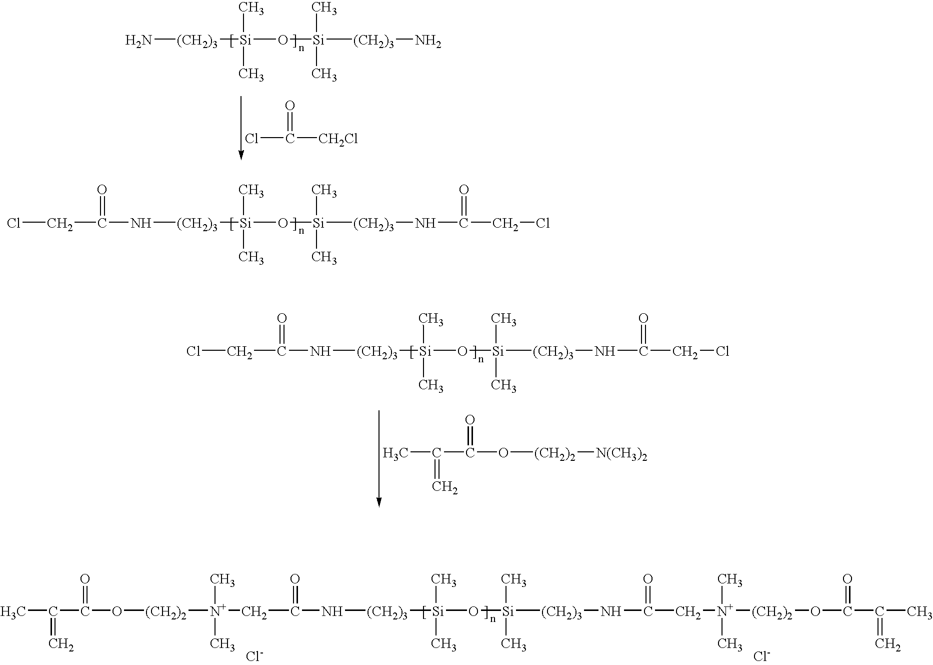

Another class of examples of applicable cationic silicon-containing monomeric units for use herein include cationic monomers of formula XXII: 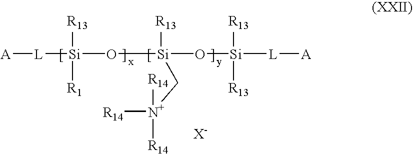
wherein x is 0 to 1000, y is 1 to 300, each L can be the same or different and is as defined above for L in formula I; X⁻ is at least a single charged counter ion as defined above for X⁻ in formula I; each R₁, R₁₃ and R₁₄ are independently as defined above for R₁ in formula I and A is a polymerizable vinyl moiety.

A preferred cationic random copolymer of formula XXII is shown in formula XXIII below: 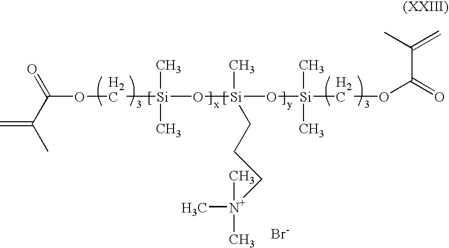
wherein x is 0 to 1000 and y is 1 to 300.

A schematic method for making the cationic silicon-containing random copolymers of Formulae XXII and XXIII is provided below:

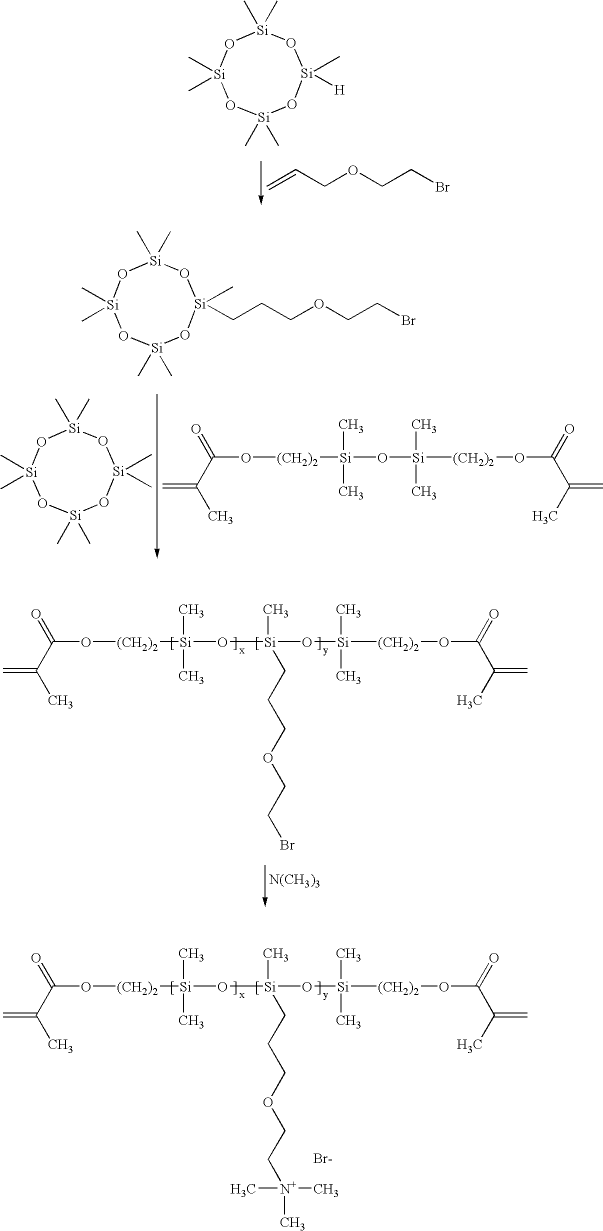

Another class of examples of applicable cationic materials for use herein include cationic random copolymers of formula XXIV: 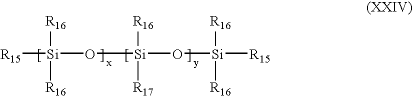
wherein x is 0 to 1000, y is 1 to 300; each R₁₅ and R₁₆ can be the same or different and can be the groups as defined above for R₁ in formula I; R₁₇ is independently one or more of the following formulae XXV and XXVI: 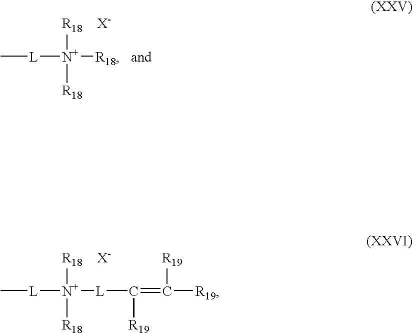
wherein L can be the same or different and is as defined above for L in formula I; X⁻ is at least a single charged counter ion as defined above for X⁻ in formula I; R₁₈ can be the same or different and can be the groups as defined above for R₁ in formula I; and R₁₉ is independently hydrogen or methyl.

A schematic representation of a synthetic method for preparing cationic silicon-containing random copolymers such as poly(dimethylsiloxane) bearing pendant polymerizable cationic groups disclosed herein is provided below.

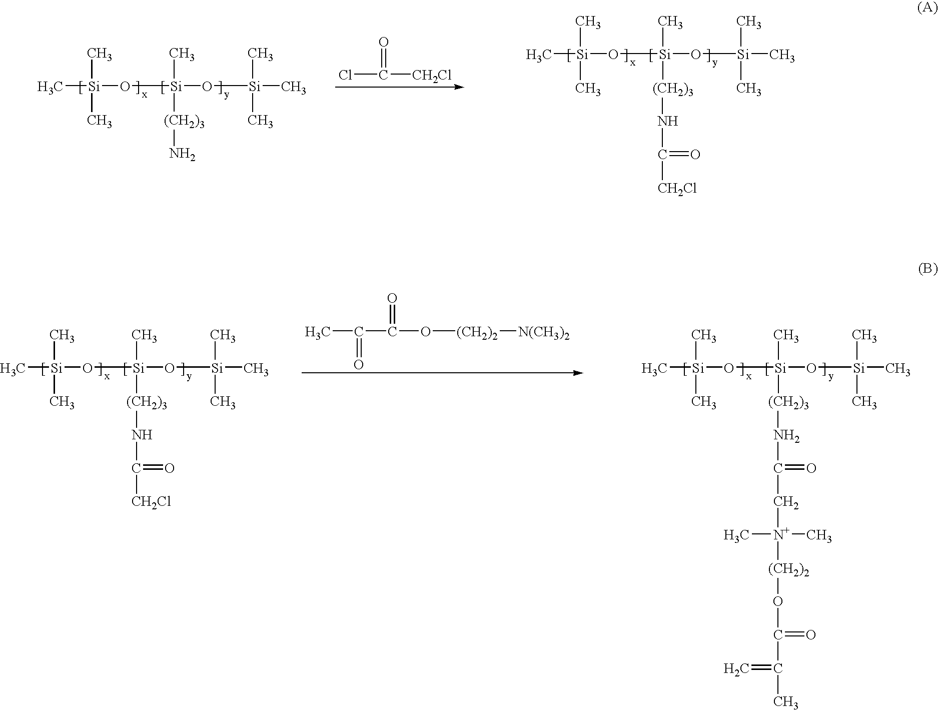

Another synthetic scheme for preparing poly(dimethylsiloxane) bearing pendant cationic groups and pendant polymerizable cationic groups is provided below.

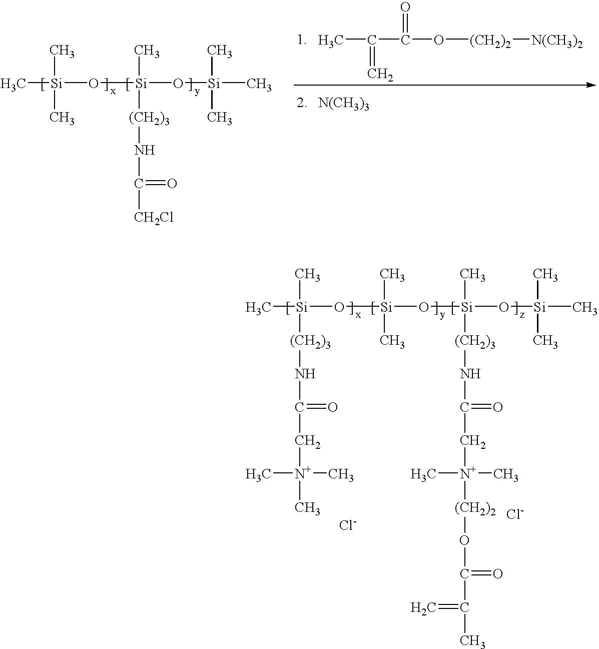

Yet another synthetic scheme for preparing poly(dimethylsiloxane) bearing pendant polymerizable groups and pendant cationic groups is provided below.

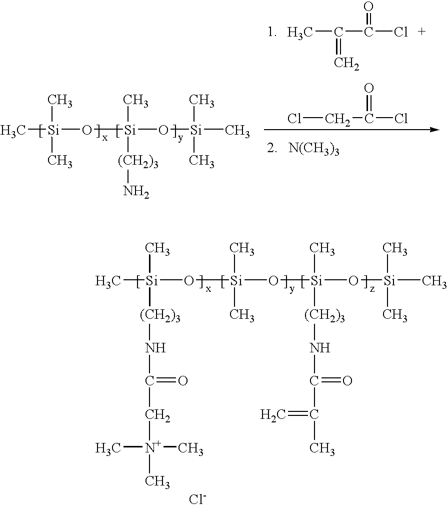

The silicone hydrogel contact lenses are packaged in a container that includes a receptacle portion to hold the contact lens and a sterile packaging solution. Examples of the container are conventional contact lens blister packages. This receptacle, containing the contact lens immersed in the solution, is hermetically sealed, for example, by sealing lidstock on the package over the receptacle. For example, the lidstock is sealed around a perimeter of the receptacle.

The solution and the contact lens are sterilized while sealed in the package receptacle. Examples of sterilization techniques include subjecting the solution and the contact lens to thermal energy, microwave radiation, gamma radiation or ultraviolet radiation. A specific example involves heating the solution and the contact lens, while sealed in the package container, to a temperature of at least 100° C., more preferably at least 120° C., such as by autoclaving.

The invention recognized the problem that silicone hydrogel contact lenses have a greater tendency than conventional, non-silicone hydrogels lenses to undergo changes in physical properties while stored in their package. Important physical properties of silicone hydrogel contact lenses include: mechanical properties, such as modulus and tear strength; water content; oxygen permeability; and surface characteristics, especially when the lens includes a surface coating. Additionally, this invention recognized that changes in mechanical properties can further result in undesired changes in dimensions of the lens, such as lens diameter.

As an example, silicone hydrogel contact lenses have a greater tendency to undergo changes in crosslinking density over time. This can lead to changes in mechanical properties, particularly modulus, and result in a shorter shelf life of the packaged lens than desired.

The problem with changes in physical properties may be more prevalent with silicone hydrogel copolymers comprising an ionic lens-forming monomer. Examples of anionic lens-forming monomers are acids, including carboxylic acid-containing monomers such as (meth)acrylic acid, itaconic acid, styrenecarboxylic acid and N-vinyloxycarbonyl-β-alanine. Examples of cationic lens-forming monomers are quaternary-ammonium containing monomers. Examples of zwitterionic lens-forming monomers are monomers containing both anionic moieties and cationic moieties. It is believed the ionic functional groups, particularly those containing carboxyl groups, have the ability to partially hydrolyze silicone-containing moieties over time, thus leading to changes in physical properties of the silicone hydrogel copolymer. And although silicone hydrogel copolymers comprising an ionic monomer are more prone to hydrolysis, even silicone hydrogel copolymers lacking such an ionic monomer may still be subject to hydrolysis and changes in physical properties when packaged and stored for prolonged periods of time, especially silicone hydrogel copolymers including a silicone-containing crosslinking agent.

In the case of silicone hydrogel copolymers comprising an ionic lens-forming monomer, one class of stabilizing agents includes agents that form an ionic complex with the hydrogel copolymer.

Thus, for silicone hydrogel copolymers comprising an anionic lens-forming monomer, stabilizing agents include agents containing a cationic charge, including cationic and zwitterionic agents that form an ionic complex with the anionic lens-forming monomer. Examples of such cationic stabilizing agents include quaternary ammonium containing materials, such as cationic cellulose, cationic guar, and chitosan derivatives containing quaternary ammonium substitution.

For silicone hydrogel copolymers comprising a cationic lens-forming monomer, stabilizing agents include agents containing an anionic charge, including anionic agents and zwitterionic agents that form an ionic complex with the cationic lens-forming monomer. Examples of such anionic stabilizing agents include polymers of (meth)acrylic acid, itaconic acid, hydroxyalkyl phosphonate and ethylenediaminetetraacetic acid. Examples of zwitterionic agents include diglycine and 3-(N-morpholino)propanesulfonic acid (MOPS).

For silicone hydrogel copolymers comprising a zwitterionic lens-forming monomer, the stabilizing agent may be a mixture of a cationically charged agent and an anionically charged agent.

A class of suitable stabilizing agents includes amine-containing agents, particularly non-polymeric amine-containing agents, such as amino hydrocarbons; amino alcohols, including monoethanolamine, diethanolamine, tris(hydroxymethyl)-aminomethane (Tris), bis-Tris and bis-Tris propane; N-morpholino-containing agents and amino acids and derivatives thereof. These agents form a complex with, and are effective at stabilizing, or inhibiting hydrolysis of, various silicone hydrogel copolymers, especially those containing an anionic lens-forming monomer such as acid-containing monomers.

The silicone hydrogel copolymer may be stabilized by mechanisms other than formation of an ionic complex, such as stabilization by hydrogen bonding of the stabilizing agent with the silicone hydrogel copolymer. As an example, stabilizing agents that will form hydrogen bonding complexes with ionic groups of the silicone hydrogel copolymer include: poly(vinylpyrrolidinone)s; poly(ethylene glycol)s; poly(vinyl alcohol)s; poly(propylene glycol)s; saccharides, including poly(saccharide)s and non-ionic celluloses and guars; polyhydric alcohols, such as propylene glycol and glycerin; and block copolymers of ethylene oxide and propylene oxide.

As another example, silicone hydrogel copolymers that are not ionically charged may be stabilized with various ionic stabilizing agents. For example, silicone hydrogel copolymers containing poly(vinylpyrrolidinone); poly(ethylene oxide), or poly(vinyl alcohol), particularly at their surfaces, may be stabilized with an anionic agent, such as polymers of acrylic acid. For example, a lens surface containing bound or entrapped poly(N-vinyl-2-pyrrolidone) can form a hydrogen-bonded complex with polymers of (meth)acrylic acid.

The stabilizing agent is included in an amount effective to inhibit changes in physical properties of the silicone hydrogel copolymer while packaged and stored. Preferably, the stabilizing agent is effective at inhibiting changes of the modulus of the silicone hydrogel copolymer to no more than 25 percent, throughout a period of at least 1 year, more preferably at least 2 years, and most preferably for at least 3 years, when stored at room temperature (25° C.). Preferably, the stabilizing agent is effective at inhibiting changes in water content to less than 1 weight percent, more preferably less than 0.5 weight percent, when stored for these periods of time at room temperature. Preferably, the stabilizing agent is effective at inhibiting changes in lens diameter of less than 0.1 micron, more preferably less than 0.05 micron, when stored for these periods of time at room temperature.

Stability of a silicone hydrogel contact lens may be tested using methods known in the art for testing shelf life of a silicone hydrogel lens stored in its solution and package. One manner of such testing is on a “real-time” basis, where one or more lots of contact lenses are stored at room temperature with several lenses tested at various time intervals. If the lenses maintain their physical properties at a tested time interval, then the lens has the desired stability for that time interval. Another manner of such testing is on an “accelerated” basis, following FDA (U.S. Food and Drug Administration) guidelines for accelerated shelf life testing. As a first example, the lots of contact lenses are stored at 45° C. with several lenses tested at various time intervals; in this case, estimated stability time corresponds to four times the test interval. As a second example, the lots of contact lenses are stored at 60° C. with several lenses tested at various time intervals; in this case, estimated stability corresponds to 11.3 times the test interval. Thus, in order to determine if a lens is stable for 3 years, the test interval under this accelerated testing method would be 97 days; to determine if a lens is stable for 1 years, the test interval under this accelerated testing method would be 33 days. Such tests are generally conducted at 45% relative humidity.

The packaging solution is an aqueous solution that includes the stabilizing agent, preferably in an amount of 0.02 to 5.0 weight percent, based on total weight of the packaging solution. The specific amount of stabilizing agent will vary depending on the agent and the copolymer, but generally, the stabilizing agent will be present in an amount within this range.

The packaging solutions preferably have a pH of about 6.0 to 8.0, more preferably about 6.5 to 7.8, and most preferably 6.7 to 7.7. Suitable buffers include monoethanolamine, diethanolamine, triethanolamine, tromethamine (tris(hydroxymethyl)aminomethane, Tris), Bis-Tris, Bis-Tris Propane, borate, citrate, phosphate, bicarbonate, amino acids, and mixtures thereof. Examples of specific buffering agents include boric acid, sodium borate, potassium citrate, citric acid, Bis-Tris, Bis-Tris Propane, and sodium bicarbonate. When present, buffers will generally be used in amounts ranging from about 0.05 to 2.5 percent by weight, and preferably from 0.1 to 1.5 percent by weight. Some of the stabilizing agents will act as buffers, and if desired, a supplemental buffering agent may be employed. It has been found that stabilization is dependent on pH, as illustrated in the accompanying examples.

The packaging solutions may further include a tonicity adjusting agent, optionally in the form of a buffering agent, for providing an isotonic or near-isotonic solution having an osmolality of about 200 to 400 mOsm/kg, more preferably about 250 to 350 mOsm/kg. Examples of suitable tonicity adjusting agents include sodium and potassium chloride, dextrose, glycerin, calcium and magnesium chloride. When present, these agents will generally be used in amounts ranging from about 0.01 to 2.5 weight percent and preferably from about 0.2 to about 1.5 weight percent.

Optionally, the packaging solutions may include an antimicrobial agent, but it is preferred that the solutions lack such an agent.

The following examples illustrate various preferred embodiments of this invention.

A monomer mixture was prepared by mixing the following components: M₂D₃₉, a monomer of formula (XIX) where n is about 39; N-vinyl-2-pyrrolidone (NVP); tris(trimethylsiloxy)silylpropyl methacrylate (Tris); 2-hydroxyethyl methacrylate (Hema); a diluent, propylene glycol; a UV blocker, 2-(3-(2H-benzotriazol-yl)-4-hydroxy-phenyl)ethyl methacrylate; Vaso-64 initiator; and tint agent, 1,4-bis[4-(2-methacryloxyethyl)phenylamino]anthraquinone. The mixture was added to a two-part polypropylene mold, including a posterior mold half for forming the posterior contact lens surface, and an anterior mold half for forming the anterior mold half. The mixture was cured thermally while contained in the mold. The resultant contact lenses were removed from the mold, extracted and hydrated.

The buffers listed in Table 1 were prepared. The borate buffers include boric acid and sodium borate, with the ratio of these components adjusted to obtain the desired pH value. The phosphate buffers include sodium phosphate monobasic and sodium phosphate dibasic, with the ratio of these components adjusted to obtain the desired pH value. The citrate buffers include sodium citrate, with HCl added as necessary to obtain the desired pH value. The Trizma buffers include Trizma (tromethamine), with HCl added as necessary to obtain the desired pH value. The MOPS buffers include 3-(N-morpholino)propanesulfonic acid, with NaOH added as necessary to obtain the desired pH value. The diglycine buffers include diglycine, with NaOH added as necessary to obtain the desired pH value.

TABLE 1

| Composition | Buffer/pH |
|---|---|
| A | Borate 6.7 |
| B | Borate 7.2 |
| C | Borate 7.7 |
| D | Phosphate 6.7 |
| E | Phosphate 7.2 |
| F | Phosphate 7.7 |
| G | Citrate 6.7 |
| H | Citrate 7.2 |
| I | Citrate 7.7 |
| J | Trizma 6.7 |
| K | Trizma 7.2 |
| L | Trizma 7.7 |
| M | MOPS 6.7 |
| N | MOPS 7.2 |
| O | MOPS 7.7 |
| P | Diglycine 6.7 |
| Q | Diglycine 7.2 |
| R | Diglycine 7.7 |

As controls, various properties of contact lenses of Example 1 were measured, including water content (wt % water), diameter (mm), and modulus (g/mm²). Contact lenses of Example 1 were immersed in each of the buffers in Table 1 in a contact lens glass vial package. The packages were sealed with lidstock, and then autoclaved for 30 minutes at 121° C., either for one cycle or two cycles (designated by 1× or 2×, respectively, in the following tables). Properties of the sample contact lenses were remeasured following the autoclave cycle(s). Modulus tests were conducted according to ASTM D-1708a, employing an Instron (Model 4502) instrument where the hydrogel sample is immersed in borate buffered saline; an appropriate size of the film sample is gauge length 22 mm and width 4.75 mm, where the sample further has ends forming a dogbone shape to accommodate gripping of the sample with clamps of the Instron instrument, and a thickness of 200±50 microns. Water content is measured by comparing the weight of a hydrogel contact lens in its hydrated and dehydrated states. Average values are reported in the following tables.

TABLE 2

| | | Modulus | Modulus | |
|---|---|---|---|---|
| | Buffer/pH | (1×) | (2×) | Change |
| A | Borate 6.7 | 121.0 | 126.0 | 5.0 |
| B | Borate 7.2 | 135.0 | 150.0 | 15.0 |
| C | Borate 7.7 | 165.0 | 234.0 | 69.0 |
| D | Phosphate 6.7 | 125.0 | 129.0 | 4.0 |
| E | Phosphate 7.2 | 167.0 | 269.0 | 102.0 |
| F | Phosphate 7.7 | 278.0 | 569.0 | 291.0 |
| G | Citrate 6.7 | 142.0 | 206.0 | 64.0 |
| H | Citrate 7.2 | 270.0 | 407.0 | 137.0 |
| I | Citrate 7.7 | 466.0 | 581.0 | 115.0 |
| J | TRIZMA 6.7 | 185.0 | 182.0 | −3.0 |
| K | TRIZMA 7.2 | 177.0 | 162.0 | −15.0 |
| L | TRIZMA 7.7 | 149.0 | 158.0 | 9.0 |
| M | MOPS 6.7 | 177.0 | 164.0 | −13.0 |
| N | MOPS 7.2 | 157.0 | 140.0 | −17.0 |
| O | MOPS 7.7 | 120.0 | 129.0 | 9.0 |
| P | Diglycine 6.7 | 157.0 | 143.0 | −14.0 |
| Q | Diglycine 7.2 | 163.0 | 149.0 | −14.0 |
| R | Diglycine 7.7 | 146.0 | 147.0 | 1.0 |

TABLE 3

| | | % Water | % Water | % Water |
|---|---|---|---|---|
| | Buffer/pH | 1× | 2× | Change |
| A | Borate 6.7 | 50.2 | 50.5 | 0.3 |
| B | Borate 7.2 | 49.8 | 51.3 | 1.5 |
| C | Borate 7.7 | 53.1 | 52.0 | −1.1 |
| D | Phosphate 6.7 | 50.5 | 50.1 | −0.4 |
| E | Phosphate 7.2 | 52.8 | 49.7 | −3.1 |
| F | Phosphate 7.7 | 50.5 | 49.7 | −0.8 |
| G | Citrate 6.7 | 51.0 | 50.3 | −0.7 |
| H | Citrate 7.2 | 51.0 | 49.3 | −1.7 |
| I | Citrate 7.7 | 50.6 | 48.1 | −2.5 |
| J | TRIZMA 6.7 | 48.0 | 47.5 | −0.5 |
| K | TRIZMA 7.2 | 47.7 | 47.9 | 0.2 |
| L | TRIZMA 7.7 | 48.0 | 48.6 | 0.6 |
| M | MOPS 6.7 | 50.7 | 48.4 | −2.3 |
| N | MOPS 7.2 | 48.4 | 48.6 | 0.2 |
| O | MOPS 7.7 | 50.2 | 51.1 | 0.9 |
| P | Diglycine 6.7 | 49.5 | 47.6 | −1.9 |
| Q | Diglycine 7.2 | 48.2 | 49.2 | 1.0 |
| R | Diglycine 7.7 | 48.7 | 49.0 | 0.3 |

TABLE 4

| | | 1× | std. | 2× | std. | |
|---|---|---|---|---|---|---|
| | Buffer/pH | diameter | dev. | diameter | dev. | Change |
| A | Borate 6.7 | 13.527 | 0.085 | 13.580 | 0.030 | 0.053 |
| B | Borate 7.2 | 13.538 | 0.067 | 13.525 | 0.083 | −0.013 |
| C | Borate 7.7 | 13.609 | 0.054 | 13.351 | 0.044 | −0.258 |
| D | Phosphate 6.7 | 13.661 | 0.104 | 13.583 | 0.040 | −0.078 |
| E | Phosphate 7.2 | 13.524 | 0.081 | 13.208 | 0.089 | −0.316 |
| F | Phosphate 7.7 | 13.334 | 0.101 | 12.891 | 0.084 | −0.443 |
| G | Citrate 6.7 | 13.571 | 0.047 | 13.361 | 0.057 | −0.209 |
| H | Citrate 7.2 | 13.400 | 0.048 | 13.051 | 0.074 | −0.350 |
| I | Citrate 7.7 | 13.223 | 0.113 | 12.918 | 0.123 | −0.305 |
| J | TRIZMA 6.7 | 13.415 | 0.027 | 13.398 | 0.032 | −0.017 |
| K | TRIZMA 7.2 | 13.421 | 0.040 | 13.391 | 0.030 | −0.030 |
| L | TRIZMA 7.7 | 13.466 | 0.031 | 13.480 | 0.021 | 0.014 |
| M | MOPS 6.7 | 13.451 | 0.030 | 13.440 | 0.027 | −0.011 |
| N | MOPS 7.2 | 13.487 | 0.022 | 13.515 | 0.032 | 0.028 |
| O | MOPS 7.7 | 13.565 | 0.066 | 13.619 | 0.041 | 0.055 |
| P | Diglycine 6.7 | 13.404 | 0.035 | 13.418 | 0.029 | 0.014 |
| Q | Diglycine 7.2 | 13.434 | 0.056 | 13.409 | 0.028 | −0.025 |
| R | Diglycine 7.7 | 13.476 | 0.036 | 13.479 | 0.047 | 0.003 |

The comparison of lenses subjected to one and two autoclave cycles is useful for screening packaging solutions, i.e., packaging solutions that result in lenses exhibiting significant changes in a physical property between one and two autoclave cycles are unlikely to result in contact lenses remaining stable for extended periods. As seen in Tables 2-4, many of the packaging solutions that did not contain a stabilizing agent of this invention resulted in unacceptable stability of the contact lens packaged therein.

Various solutions and contact lenses were tested on an accelerated shelf life basis, following USFDA guidelines for such accelerated shelf life testing. Contact lenses immersed in a packaging solution of Table 1 were stored at 60° C. with several lenses tested at various time intervals; in this case, estimated stability corresponds to 11.3 times the test interval.

TABLE 5

| | | | Diameter | | ▴D |
|---|---|---|---|---|---|
| | Baseline | | 16 day | | (Base- |
| Buffer/pH | Diameter | Std. Dev | (60 C.) | Std. Dev. | 16 day) |
| Borate 7.2 | 13.608 | 0.038 | 13.398 | 0.064 | −0.210 |
| Trizma 7.2 | 13.434 | 0.021 | 13.441 | 0.023 | 0.007 |
| MOPS 7.2 | 13.447 | 0.040 | 13.513 | 0.029 | 0.066 |
| Diglycine 7.2 | 13.422 | 0.024 | 13.450 | 0.019 | 0.029 |

TABLE 6

| | | | Diameter | | ▴D |
|---|---|---|---|---|---|
| | Baseline | | 35 Day | | (Base- |
| Buffer/pH | Diameter | Std. Dev | (60 C.) | Std. Dev. | 35 day) |
| Borate 7.2 | 13.608 | 0.038 | 13.138 | 0.078 | −0.470 |
| Trizma 7.2 | 13.434 | 0.021 | 13.490 | 0.029 | 0.055 |
| MOPS 7.2 | 13.447 | 0.040 | 13.468 | 0.028 | 0.021 |
| Diglycine 7.2 | 13.422 | 0.024 | 13.477 | 0.034 | 0.055 |

TABLE 7

| | | | Modulus | | |
|---|---|---|---|---|---|
| | Baseline | Std. | 16 day | Std. | Change in |
| Buffer/pH | Modulus | Dev | (60 C.) | Dev. | Modulus |
| Borate 7.2 | 131 | 12 | 229 | 32 | 98 |
| Trizma 7.2 | 163 | 16 | 134 | 9 | −29 |
| MOPS 7.2 | 162 | 10 | 134 | 6 | −28 |
| Diglycine 7.2 | 166 | 9 | 132 | 13 | −34 |

As seen in Tables 5-7, packaging solutions containing a stabilizing agent of this invention were more effective at stabilizing the contact lens than the comparative solution (borate 7.2).

Having thus described various preferred embodiment of the invention, those skilled in the art will appreciate that various modifications, additions, and changes may be made thereto without departing from the spirit and scope of the invention, as set forth in the following claims.

We claim:

1. A contact lens package including a sealed receptacle containing a contact lens immersed in a sterile solution, wherein the contact lens is made of a silicone hydrogel copolymer, and the solution comprises a stabilizing agent in an amount effective to inhibit changes in physical properties of the silicone hydrogel copolymer.

2. The package of claim 1, wherein the silicone hydrogel copolymer is ionic.

3. The package of claim 2, wherein the silicone hydrogel copolymer is anionic.

4. The package of claim 2, wherein the silicone hydrogel copolymer is cationic.

5. The package of claim 2, wherein the stabilizing agent is an amine that complexes with anionic groups of the copolymer.

6. The package of claim 5, wherein the stabilizing agent is selected from the group consisting of: amino hydrocarbons; amino alcohols; and amino acids.

7. The package of claim 3, wherein the stabilizing agent is a quaternary ammonium-containing compound that forms an ionic complex with anionic groups of the copolymer.

8. The package of claim 4, wherein the stabilizing agent includes anionic groups that complex with the cationic groups of the copolymer.

9. The package of claim 2, wherein the stabilizing agent contains groups that hydrogen bond with the ionic groups of the copolymer.

10. The package of claim 9, wherein the stabilizing agent is selected from the group consisting of: poly(vinylpyrrolidinone)s; poly(ethylene glycol)s; poly(vinyl alcohol)s; poly(propylene glycol)s; saccharides; and polyhydric alcohols.

11. The package of claim 1, wherein the silicone hydrogel copolymer is the polymerization product of a monomeric mixture comprising: a silicone-containing crosslinking monomer; and a hydrophilic monomer.

12. The package of claim 11, wherein the silicone hydrogel copolymer is the polymerization product of a monomeric mixture comprising: the silicone-containing crosslinking monomer; the hydrophilic monomer; and a monofunctional silicone-containing monomer.

13. The package of claim 2, wherein the silicone hydrogel copolymer is the polymerization product of a monomeric mixture comprising: a silicone-containing monomer; a hydrophilic monomer; and an ionic monomer.

14. The package of claim 13, wherein the ionic monomer comprises a cationic monomer, and the stabilizing agent is selected from the group consisting of MOPS, tromethamine, diglycine, and mixtures thereof.

15. The package of claim 1, wherein the solution has a pH of 6 to 8.

16. The package of claim 1, wherein the solution lacks an antimicrobial agent.

17. The package of claim 1, wherein the solution comprises 0.02 to 5.0 weight percent of the stabilizing agent.

18. The package of claim 1, wherein lidstock is sealed over the receptacle containing the solution and the contact lens.

19. The package of claim 18, wherein the lidstock is sealed around a perimeter of the receptacle.

20. The package of claim 18, wherein the solution and the contact lens are subjected to thermal energy while sealed in the package receptacle.

21. The package of claim 20, wherein the solution and the contact lens are heated to a temperature of at least 100° C.

22. The package of claim 18, wherein the solution and the contact lens are sterilized while sealed in the package receptacle.

23. The package of claim 1, wherein the package containing the solution and the contact lens is autoclaved.

24. The package of claim 1, wherein the stabilizing agent is in an amount effective to inhibit changes in modulus of the silicone hydrogel copolymer.

25. A method comprising:
sealing a receptacle of a contact lens package that contains a solution and a contact lens, wherein the contact lens is made of a silicone hydrogel copolymer; and
storing the contact lens in the package for an extended period of time, wherein the stabilizing agent inhibits changes in physical properties of the silicone hydrogel copolymer during storage.

26. A method of improving the hydrolytic stability of a contact lens made of a silicone hydrogel copolymer, comprising storing the contact lens in a sealed package and immersed in a sterile solution comprising a stabilizing agent, wherein the stabilizing agent inhibits changes in physical properties of the silicone hydrogel copolymer during storage.

27. A method of increasing the shelf life of a contact lens made of a silicone hydrogel copolymer and contained in a sealed package, comprising storing the contact lens in the sealed package and immersed in a solution comprising a stabilizing agent that inhibits changes in physical properties of the silicone hydrogel copolymer during storage.

28. A method of providing a silicone hydrogel contact lens with a shelf life of at least 3 years, comprising storing the contact lens in the sealed package and immersed in a solution comprising a stabilizing agent that inhibits changes in physical properties of the silicone hydrogel copolymer during storage.
